(19) United States
(12) Patent Application Publication
(10) Pub. No.: US 2003/0170635 A1
(43) Pub. Date: Sep. 11, 2003

(54) Slotted electrophoresis gel composition and methods of use thereof

(75) Inventors: Philip W. Hammond, Brighton, MA (US); Christopher P. Adams, Somerville, MA (US); Ezra S. Abrams, West Newton, MA (US); T. Christian Boles, Waltham, MA (US)

Correspondence Address: TESTA, HURWITZ & THIBEAULT, LLP, HIGH STREET TOWER, 125 HIGH STREET, BOSTON, MA, 02110, US

(73) Assignee: EXACT Sciences Corporation

(21) Appl. No.: 10/087,221

(22) Filed: Mar. 1, 2002

Related U.S. Application Data
(63) Continuation of application No. 09/206,173, filed on Dec. 4, 1998, now abandoned.
(60) Provisional application No. 60/067,556, filed on Dec. 5, 1997.

(51) Int. Cl. C12Q 1/68; G01L 1/20

(52) U.S. Cl. 435/6; 204/461

(57) ABSTRACT

A slotted electrophoresis gel composition and methods of use are disclosed in the present invention. The invention also describes an apparatus that is used to produce a slotted electrophoresis gel composition.

### RELATED APPLICATION

This application is claiming the benefit of Provisional Application No. 60/067,556, filed on Dec. 5, 1997, the entire teachings of which are incorporated herein by reference.

### BACKGROUND OF THE INVENTION

A biological sample can be composed of a heterogenous group of components, for example, proteins or nucleic acids. It is often desirable to separate such components contained within a biological sample. Electrophoresis is a commonly applied method to perform such a task. Components of a sample can be selectively separated from other constituents based upon their size and/or charge density using an electric field in conjunction with a solid support matrix such as an electrophoretic gel. Typically, the gel is comprised of a continuous composition of material such as polyacrylamide, starch or agarose. However, there are occasions when multilayered gels are envisaged as a means for examining a particular experimental phenomenon. There are inherent difficulties attendant to the formation of multilayered gels as now practiced. It can be time consuming and tedious to produce such a gel. Defining precise regions for individual layers can be difficult at best. There exists a need to produce reliable and easy to use gels that have the ability to form a multilayered gel system

### SUMMARY OF THE INVENTION

The present invention pertains to a slotted electrophoresis gel composition comprising a primary gel and a secondary gel. The primary gel comprises a first matrix comprising polyacrylamide, agarose, starch or a combination thereof. The secondary gel comprises a second matrix comprising polyacrylamide, agarose, starch or a combination thereof. The primary gel can comprise a sample loading zone and additionally comprises one, or more, slots where each slot comprises an opening suitable for filling with a secondary gel. The secondary gel can comprise matrix material that differs from the primary gel. Alternatively, the secondary gel, in addition to containing matrix material, can also be comprised of one, or more, immobilized probes which are specific for a target molecule. For example, the primary gel can comprise a 12% polyacrylamide solution suitable for electrophoresis and the secondary gel can comprise a 12% polyacrylamide solution containing specific nucleic acid probe sequences immobilized wherein the probes are copolymerized upon polymerization of the solution. Upon copolymerization the probes are immobilized within the secondary gel, thus creating a multilayered gel composition comprising a primary gel and one, or more, secondary gels. The slotted electrophoresis gel composition of the present invention can be employed to isolate and/or detect the presence, or absence, of a target molecule contained within a biological sample. The biological sample can be from any source and can contain any molecule that is amenable to electrophoresis.

In one embodiment, the invention pertains to an electrophoresis gel composition comprising a primary gel that has a sample loading zone at the top of the gel and at least one preformed slot comprising an opening for a secondary gel. The slot, or slots, can have any geometric configuration within the primary gel. The slots can also have any geometric shape such as a square, rectangle, circular or combinations thereof. The slot's thickness can be the same or less than the thickness of the primary gel. The slot opening can be filled using a secondary gel. The secondary gel can differ from the primary gel in terms of matrix material. Additionally, the secondary gel can contain immobilized probes specific for a target molecule.

In another embodiment, the invention relates to an apparatus that can be used in forming slotted gels for electrophoresis. The slot-forming apparatus comprising at least one slot-forming element that can be used to form one, or more, slots in the primary electrophoresis gel. The elements can be attached to, or contiguous with, a horizontal cross-piece to facilitate placement and removal from the gel casting apparatus. For example, a slot-forming element can comprise a comb-like” structure so that all elements can be inserted and removed in a single operation. Preferably, the slot-forming comb comprises one, or more, elements that can be elongated and project into the primary gel. These elements are used to form slots within the primary gel during the casting of that gel. The slot-forming elements can have a width from about 0.2 to about 20.0 mm. Preferably, the width of the slot-forming element is from about 0.5 to about 5.0 mm Typically, when the slot-forming elements are removed from the primary gel, depending upon the geometric shape of the element, for example rectangular, a column-like opening will occur in the primary gel. The column-like opening, or openings, can the be filled using a secondary gel or even buffer if, for example, the slotted gel were to be stored under suitable conditions.

In another embodiment of the invention, a method for detecting the presence, or absence, of one, or more, target molecules in a biological sample is encompassed. The method described herein employs a slotted electrophoresis gel composition. The electrophoresis gel can contain one, or more, preformed slots positioned within the primary gel. At least one slot can comprise a secondary gel containing one, or more, immobilized probes which are used to specifically interact with and capture the target molecule, if present in the biological sample. The probe can be specific for nucleic acids, polypeptides, proteins, carbohydrates, lipids or combinations, analogs and modifications thereof. After a suitable amount of time, under suitable conditions, the electrophoresis is stopped and the presence of the target molecules in the slots are detected. The detection of a target molecule in a slot comprising a secondary gel containing a probe specific for the target molecule is indicative of the presence of the target molecule in the biological sample.

In one embodiment of the invention, a method for isolating and detecting one, or more, target molecules in a biological sample is described using a slotted electrophoresis gel composition. The primary gel is comprised of a gel matrix material used to form a solid support for the separation of molecules. A slot, or slots, are contained within the primary gel and comprise a secondary gel which differs in matrix material from that of the primary gel in that the percentage of matrix differs or that a different matrix material is employed. Mobilities of molecules contained within a sample can be selectively altered by electrophoresis through one or more slots comprising a secondary gel. Passage through the slot(s) can augment the separation, isolation and detection of target molecules if they interact in a different manner, or to a different extent, with the secondary gel comprising a particular slot within the primary gel. After a suitable amount of time, under suitable conditions, the electrophoresis is stopped and the presence of the target molecules in the slots are detected. The detection of a target molecule in the electrophoretic matrix, either the primary or secondary gel, is indicative of the presence of the target molecule in the biological sample.

The invention also pertains to a diagnostic kit used to detect the presence, or absence, of a target molecule in a biological sample. The diagnostic kit contains a slotted electrophoresis gel composition comprising a primary gel that has a slot-forming comb inserted into the primary gel perpendicular or opposite to the sample loading zone wherein at least one preformed slot opening for a secondary gel is formed upon removal of the comb. Optionally, a sample well comb is inserted into the top edge of the primary gel which forms a sample loading zone upon removal of the comb. The secondary gel can be comprised of a reagent, or reagents, used for detecting the presence, or absence, of a target molecule in a biological sample. The reagent(s) used can contain a probe specific for a target molecule of interest. The probe can be immobilized to a matrix material which comprises the secondary gel. One, or more, secondary gels can be provided with the diagnostic kit or, alternatively, they can be prepared by the user.

Thus based on the compositions, apparatus and methods described herein, novel approaches that are efficient and easy to perform can be employed to detect the presence, or absence, of a target molecule in a biological sample. Multiple screening can be accomplished by employing the methods of analysis described herein. By using multiple secondary gels containing different probes specific for different target molecules, a biological sample can be screened for multiple target molecules.

### BRIEF DESCRIPTION OF THE DRAWINGS

FIG. 1 is a schematic representation of slot-forming elements.

FIG. 2a is a schematic representation of a single horizontal slot contained within a slotted electrophoresis gel.

FIG. 2b is a schematic representation of multiple horizontal slot contained within a slotted electrophoresis gel.

FIG. 2c is a schematic representation of a single vertical slot contained within a slotted electrophoresis gel.

FIG. 2d is a schematic representation of multiple vertical slot contained within a slotted electrophoresis gel.

FIG. 3 is a schematic representation of an apparatus used to produce a slotted electrophoresis gel.

FIG. 4 is a schematic representation of a method employing an apparatus for producing a slotted electrophoresis gel.

FIG. 5 is a fluorescence scan of target molecule/probe complexes using a slotted electrophoresis gel.

FIG. 6 is a fluorescence scan of a target molecule/probe complex using a slotted electrophoresis gel.

FIG. 7 is a schematic representation of two dimensional slotted gel electrophoresis.

FIG. 8 is a slotted electrophoresis gel demonstrating the results of multiple sequential displacement reactions.

### DETAILED DESCRIPTION OF THE INVENTION

The present invention pertains to slotted electrophoresis gel compositions, apparatus for making these gel compositions and methods of use thereof. The slotted electrophoresis gel compositions can be employed when multi-layered gels are desired for analysis. The slotted electrophoresis gel compositions can be used to isolate and detect the presence, or absence, of a single or multiple target molecules contained within a biological sample. The preformed slots present in the primary gel can be comprised of a secondary gel. The secondary gel that can be present in the slot, or slots, can have one, or more, probes contained therein which are specific for one, or more, target molecules of interest.

The term “matrix” as used herein refers to the immobilized polymeric components of the electrophoresis gel composition which provide the molecular sieving properties of the medium, and also provide for immobilization of probes. Examples of suitable matrix materials include gel forming polymers such as crosslinked polyacrylamide, agarose and starch

The present invention pertains to an apparatus and methods of using the apparatus to construct slotted electrophoresis gels, and to methods that describe isolating and/or detecting the presence, or absence, of a target molecule contained within a biological sample using a slotted electrophoresis gel. The methods of the present invention are applicable to the analysis of any chemical entity that is amenable to electrophoresis, such as a molecule that can assume a charge when placed in an electric field. Such chemical entities include, but are not limited to, nucleic acids, both deoxyribonucleic acid and ribonucleic acid, polypeptides, proteins, lipids, carbohydrates or combinations, modifications and analogs thereof. Small organic molecules, for example, pharmaceutical drugs, neurotransmitters, etc., are also encompassed within the scope of this invention.

The biological sample can originate from any source and can contain any molecule that is amenable to electrophoresis. Biological samples are specifically encompassed by this invention. Biological samples include, but are not limited to, bodily fluids (e.g., blood, sweat, urine, tracheal and bronchial exudate, semen and cerebral spinal fluid) and bodily tissue homogenate (e.g., skin, other organs and hair). Other biological sources can include microbial, viral, parasitic and fungi samples. Agricultural sources are also encompassed by the present invention.

The biological sample is subjected to treatment, as known to those skilled in the art, such that the target molecule contained therein is amenable to electrophoretic analysis. For example, a tissue can be homogenized under suitable conditions (e.g., appropriate buffers) to render a homogenate preparation that can then be centrifuged yielding a supernatant containing at least one target molecule of interest.

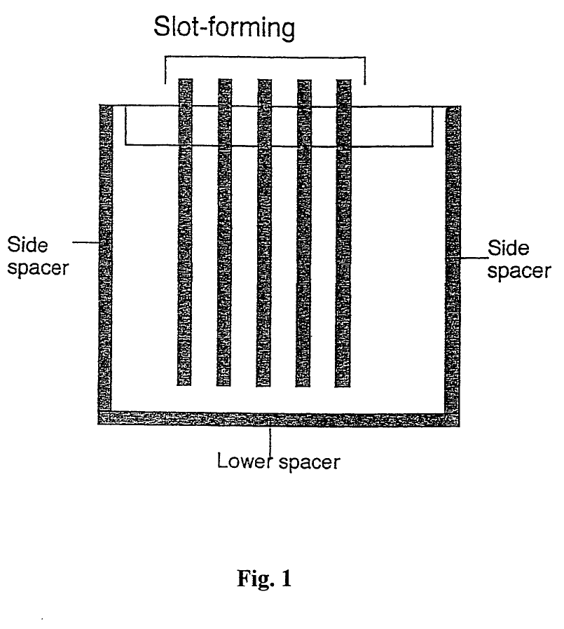

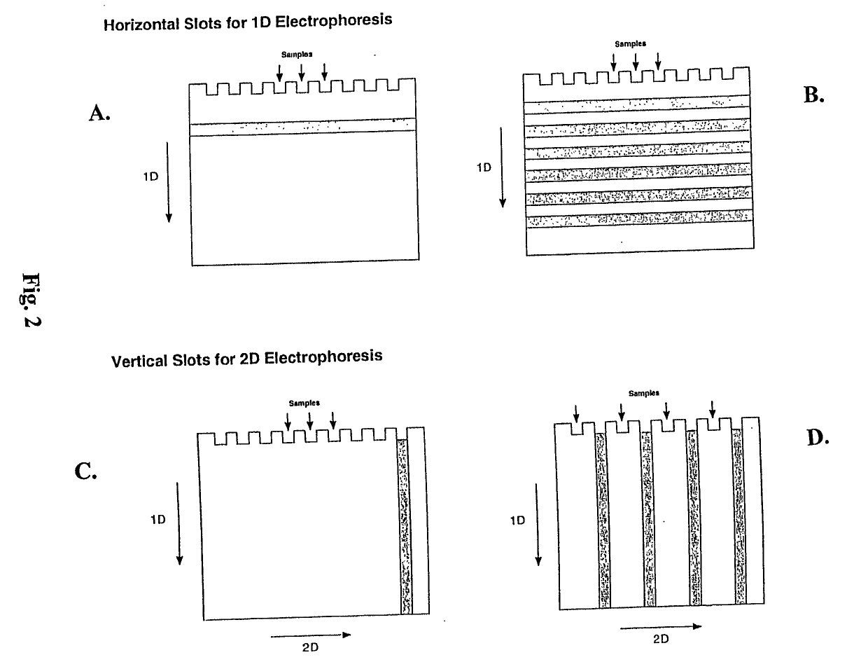

The primary gel comprises multiple layers comprising a slot, or slots, within its matrix. After the primary gel is cast, but before it polymerizes, a slot-forming comb is inserted into the primary gel. The slot-forming comb has attached to it slot-forming elements which are finger-like projection that are inserted into the primary gel. (See FIG. 1). The slot, or slots, can have any geometric configuration in the gel. For example, the slot can be elongated and aligned along the longitudinal axis of the primary gel, or it can be aligned perpendicular or diagonal to the longitudinal axis of the primary gel. (See FIG. 2).

The slotted gel composition can be stored under suitable conditions with the slot element remaining in the gel, or, alternatively, with, for example, suitable buffer in the slots until the gel composition is ready for use.

Typically, the slot comprises a secondary gel of matrix material. In one embodiment, the secondary gel of matrix material can be different from that used to form the primary gel. For example, if the primary gel comprises 10% polyacrylamide, then the slots can contain 15% polyacrylamide or even another matrix material such as agarose. If multiple slots are produced, then multiple, different matrix materials can be used to fill in the slots respectively to form multiple layers. Mobilities of components contained within a sample can be selectively altered by electrophoresis through one or more slots of a gel having different matrix compositions from that of the primary gel. Passage through the slot(s) can augment the separation, isolation and detection of sample components, including a target molecule, if they interact in a different manner, or to a different extent, with the secondary gel comprising a particular slot within a gel.

Following polymerization of the primary gel, the slot-forming comb can be removed to allow a secondary gel to be added to the opening, or openings, created by the slot-forming elements. The secondary gel comprising matrix material that can also contain immobilized probes that will specifically interact with the target molecule, if present in the biological sample.

Once the biological sample is prepared for electrophoresis, the sample can be introduced to the gel via the sample loading zone upon removal of the sample well comb. Preferably, the sample loading zone comprises sample wells. Alternatively, samples can be deposited on the surface of a horizontal gel. (See, Allen, et al., Biotechniques, 7:736-744 (1989)). The number of wells used depends upon the sample and analysis to be performed. For example, if there are multiple samples to be analyzed, for example, ten samples, then there should be enough wells to reasonably accommodate the number of samples present, alternatively, if there are a great number of samples to be analyzed, then multiple electrophoresis procedures can be performed.

Typically, the size of the wells depends upon the size of teeth-like projections attached to the sample comb. Also, the number of samples to be analyzed can influence the type of sample comb to employ and therefore, the size of sample wells present. The sample can be introduced by any means well known to those skilled in the art, such as pipetting or syringe injection.

The sample is can be loaded once the sample comb has been removed. The sample can be loaded by methods known to those skilled in the art such as pipetting or syringe injection. The amount of sample loaded depends upon, for example, the desired concentration of sample to be used. At this point the gel should be contained in an apparatus appropriate for applying an electric current across the gel, that is, along the axis from the sample well to the opposite side (in other words, in the direction of the desired migration of the sample). The gel can be of any dimensions, for example, 10 cm×10 cm. The gel can comprise various percentages of matrix material, for example, 12% polyacrylamide.

Once the sample is loaded, electrophoresis can be initiated. Samples can be subjected to migration by employing approximately 5V/cm at approximately 25° C. for approximately one hour. These variables can change depending upon the sample and gel matrix employed. Typically, a Tris-glycine buffer is used for gel electrophoresis, for example, 1.5 M Tris.Cl with 10 mM glycine. After the sample has undergone electrophoresis, then the target molecule can be detected by any number of means described herein.

Target immobilization can be transient or stable for a substantial period of time, depending on the strength and lifetime of target molecule/immobilized probe interaction. The interaction between the target molecule and probe depends upon the species of molecules involved. For example, if the target molecule is a nucleic acid molecule and the probe is a nucleic acid sequence, then the interaction between them would be one of hybridization forming a hybridization complex; however, if the target molecule is a peptide (or polypeptide or protein) and the probe is an antibody (or antigen binding fragment), the interaction is one that is based on specificity and non-covalent bonding (e.g., hydrogen and ionic bonds). In one embodiment, the target molecule transiently interacts with one, or more, probes immobilized in the secondary gel matrix (e.g., forms an immobilized target/probe complex). In this embodiment the target molecule can interact and be released multiple times during migration through the slotted electrophoresis gel. In another embodiment, the target molecule interacts with an immobilized probe and remains associated with that probe.

In another embodiment of the invention, the secondary gel comprises matrix material, the same or different from the primary gel, and contains immobilized probes. The probe, or probes, employed are specific for the putative target molecule contained within a biological sample. The probes can be specific for either nucleic acids, polypeptides, proteins, lipids, carbohydrates or combinations, modifications and analogs thereof.

Typically, the probe can be a nucleic acid, nucleic acid binding peptide (or protein), antibody or antigen binding fragment thereof, polypeptide or protein. The immobilization of a probe(s), for example a nucleic acid, can be facilitated using acrylamide phosphoramidite that will modify either the 5′ or 3′ end with an acrylamide moiety (Acrydite™ phosphoramidite, Mosaic Technologies, Boston, Mass.; see also, U.S. Ser. No. 08/971,845; the teachings of which are incorporated herein in its entirety by reference).

A nucleic acid probe generally possess a sequence that is complementary to the target molecule which is also a nucleic acid. The term nucleic acid denotes single stranded or double stranded deoxyribonucleic acid (hereinafter “DNA”) or ribonucleic acid (hereinafter “RNA). The degree of complementarity between probe and target molecule can vary from about 65% to about 100% depending upon stringency conditions, which can be determined by those skilled in the art. (Current Protocols in Molecular Biology, Ausbel, F. M., et al. eds., vol. 1, Suppl, 26, 1991; the teachings of which are incorporated herein by reference in their entirety). The complementarity need only be sufficient enough to specifically bind the target molecule through hybridization and demonstrate the presence, or absence, of the target molecule.

Hybridization is understood herein to mean admixing of at least two polynucleotide sequences under conditions suitable such that when at least two complementary polynucleotide sequences are present, they will then form a double-stranded structure through base-pairing. Mismatches are permitted in the instant invention. Nucleotide mismatch can affect the affinity between polynucleotide sequences. The greater the mismatch between polynucleotide sequences, generally the affinity is lower between them as compared to perfectly matched polynucleotide sequences. Generally, the greater the mismatch between polynucleotide sequences the easier it is to disrupt any hybridization that exists between them. When mismatches between base pairs are present, they generally account for no more than 5% of the region of base-pairing. Preferably, the degree of complementarity between hybridization partners is from about 100% to about 95%.

Proteins and polypeptides that can form specific interactions with other molecules are also encompassed within this invention as suitable probes. Proteins (including polypeptides and peptides) can specifically interact with other proteins, polypeptides, peptides and nucleic acids (e.g., peptide nucleic acid molecules). They can also specifically interact with nucleic acids as in the case of trans-acting factors, or in the interaction between modified nucleic acids (nucleic acids containing a cysteine moiety) and proteins, polypeptides and peptides. Proteins (including polypeptides and peptides) can also interact with lipids and carbohydrates forming lipoproteins and glycoproteins, respectively. The reverse is true as well, that is, lipids and carbohydrates can interact with proteins, polypeptides and peptides. One of ordinary skill in the art can immobilize an amine containing entity to a matrix. (See, Japanese unexamined patent application No. H3[1991]-47097; the entire teachings of which are incorporated herein by reference).

The probe can also be an antibody or antigen binding fragment thereof. The antibody can be either a polyclonal or monoclonal antibody. Protocols for the production of either polyclonal or monoclonal antibodies against a target molecule are well known to those skilled in the art. (Current Protocols in Molecular Biology, Ausubel, F. M., et al. eds, vol. 2, 1991; the teachings of which are incorporated herein by reference in its entirety).

The present invention also pertains to electrophoresis that can be performed in two dimensions. For example, the sample can undergo electrophoresis in one dimension separating the molecule contained within the sample. The gel can then be turned ninety degrees in order to conduct electrophoresis in a second dimension. For example, the primary gel could comprise a single preformed slot comprising a secondary gel which contains multiple heterogenous probes that are not immobilized. When the gel undergoes electrophoresis in the second dimension, the probes migrate to their specific target molecule, if present. Two-dimensional gel electrophoresis is known to those skilled in the art. (Current Protocols in Molecular Biology, Ausubel, F. M., et al. eds, vol. 2, 1991; the teachings of which are incorporated herein by reference in its entirety).

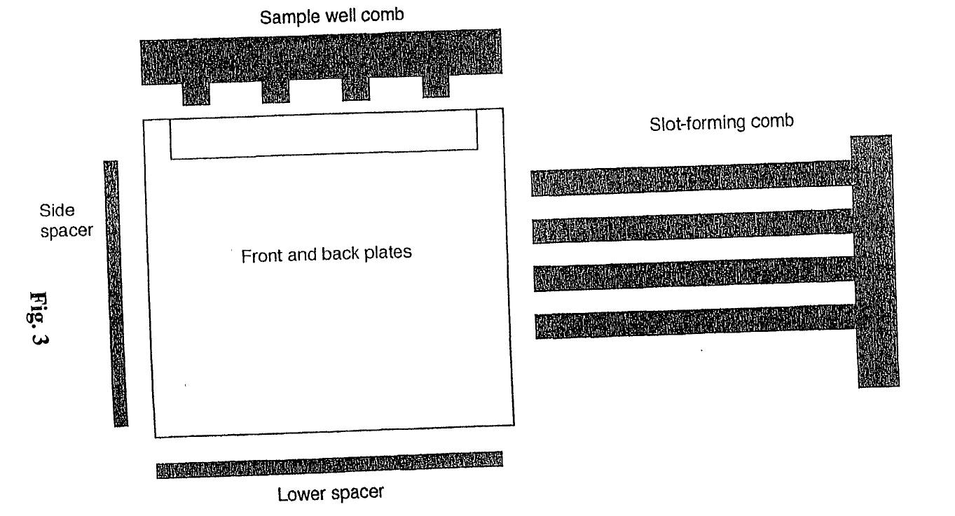

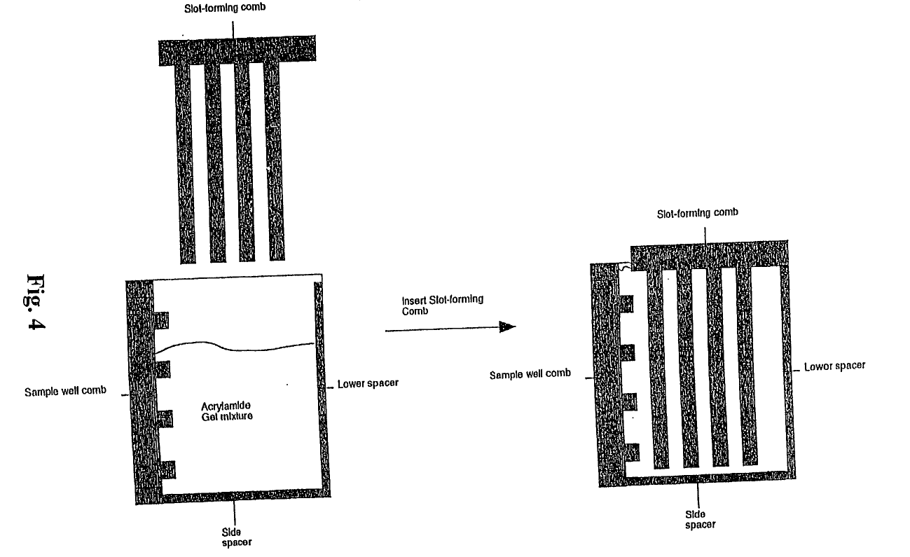

The invention also pertains to an apparatus used to produce a slotted electrophoresis gel. (See FIGS. 1, 3 and 4). The apparatus comprises at least one slot-forming comb (from about 0.2 to about 20.0 mm in width), an optional sample well comb (from about 0.4 to about 10.0 mm in width), two plates (casting plates; approximately 10 cm×10 cm) that serve to constrain the matrix material when in the liquid state, at least one spacer (from about 0.4 to about 10.0 mm in width) that is positioned between the two plates and at least two clamps that secure the two plates together. Preferably, the slots are formed by placing a slot-forming comb between the two plates prior to casting the primary gel. The casting plates can be composed of plastic, glass, steel or any other material amenable to forming an electrophoresis gel. The slot-forming comb is comprised of a slot-forming element, or elements. These elements can have any geometric configuration, for example, they can be elongated. These elements can have any dimension with respect to length, width and height. The elements can be of a composition selected from, but not limited to, plastic, steel, rubber or glass. The slot-forming elements project into the area between the two casting plates such that when the primary gel matrix material is poured, the slot-forming elements provide a penetration barrier to the matrix material. Once the primary gel matrix material has polymerized or solidified, then removal of the slot-forming comb from the apparatus will result in “slots” or openings in the primary gel devoid of primary gel matrix material. These slots within the primary gel can remain empty and be filled with a suitable buffer for storage or shipping, or they can be filled with a secondary gel of matrix material. It should be understood that if there is more than one slot in the primary gel, for example, three slots, then each slot can be filled with an unique matrix material that is different from any other matrix material used. The second, or third etc., composition matrix material can also contain immobilized probes. See Example 1.

The invention also pertains to methods for detecting the presence, or absence, of one, or more, target molecules contained within a biological sample using a slotted electrophoresis gel composition. A primary gel is produced having at least one preformed slot in any geometric configuration contained within it as described herein. A secondary gel is produced comprising a matrix material with immobilized probe, or probes, specific for a target molecule. The biological sample is prepared in a manner consistent with the notion of subjecting the preparation to electrophoresis such as that described herein. The biological sample is then introduced into the electrophoresis gel composition. This electrophoresis gel composition comprises both the primary and secondary gels. The term secondary gel refers to any slot, or slots, that comprise matrix material that is different from the matrix material used to produce the primary gel. The difference includes using the same matrix material as that used to produce the primary gel, but in addition, it contains an immobilized probe, or probes. The electrophoresis gel composition is subjected to an electric field such that the target molecule, or molecules, migrate into at least one slot containing a probe that is specific for the target molecule(s), thereby forming an immobilized target molecule/probe complex. Conditions for facilitating migration along an electrophoresis gel are well known to those skilled in the art. (Current Protocols in Molecular Biology, Ausebel, F. M., et al. eds., vols 1 & 2, 1991; the teachings of which are incorporated herein by reference in its entirety). The complex formed between the target molecule and probe is then detected.

Detection of the interaction between the target molecule and probe, for example, detection of the immobilized target molecule bound to a probe, can be accomplished in a number of different ways. For example, the target molecule can be detectably labeled prior to the interaction. Suitable labels for direct target labeling can be intensely absorbing (e.g., brightly colored), radioactive, fluorescent, phosphorescent, chemiluminescent or catalytic. Direct target labeling of nucleic acid target molecule using modified nucleotides can be accomplished by a number of enzymatic methods well known to those practiced in the art (reviewed in Sambrook, et al., “Molecular Cloning: A Laboratory Manual”, 2nd edition, Cold Spring Harbor Press, Cold Spring Harbor, N.Y. 1989; the teachings of which are incorporated herein by reference in its entirety).

Alternatively, the target molecule can be labeled indirectly using a ligand which can be recognized by a second specific binding entity which is either labeled itself or can produce a detectable signal. An example of such an indirect system is labeling using biotinylated nucleotides. In this system, the sample is labeled enzymatically using standard nucleic acid labeling techniques and biotinylated nucleotides. The resulting biotin-modified nucleic acids can be detected by the biotin-specific binding of streptavidin or avidin proteins molecules. The streptavidin or avidin molecules can be conjugated to fluorescent labels, such as fluorescein or reporter enzymes, such as alkaline phosphatase or horseradish peroxidase, which can be used to produce chemiluminescent or colorimetric signals with appropriate substrates (see Keller and Manak, “DNA Probes”, 2nd ed., Macmillan Publishers, London, 1993; Pershing, et al., eds “Diagnostic Molecular Microbiology: Principles and Applications”, American Society for Microbiology, Washington, D.C., 1993; the teachings of which are incorporated herein by reference in their entirety). Another useful detection system is the digoxygenin system which uses an anti-digoxygenin antibody, conjugated to alkaline phosphatase, which recognizes digoxygenin-dUTP incorporated into nucleic acids. (Current Protocols in Molecular Biology, Ausubel, F. M., et al. eds., vol. 1, §§ 3.18.1 to 3.19.6, 1995; the teachings of which are incorporated herein by reference in its entirety).

Detectably labeled hybridization probes can also be used as indirect target labels. For example, target nucleic acids can be indirectly labeled prior to electrophoresis by hybridization with a detectably labeled probe, hereafter termed a “sandwich” probe. The sandwich probe is designed to hybridize with a region of the target which does not overlap the region recognized by the capture probe. The sandwich probe is designed to remain associated with the target during electrophoresis, and cannot bind directly to the immobilized probe.

Sandwich probes can also be used to label target molecules after electrophoretic capture. In this labeling strategy, the unlabeled target undergoes electrophoresis and hybridized to the capture probes first. Then, the sandwich probe undergoes electrophoresis through the capture layer. In effect, the captured target now acts as a new probe for the sandwich probe. The captured target sandwich probe complex can now be detected through the sandwich probe label.

Blotting techniques can also be adapted for detection of target molecule/probe interaction. For example, a detection surface is juxtaposed to the separation medium having bound sample components, and the sample components then migrate to the detection surface, optionally assisted by, for example, chemical means such as solvent or reagent changes, where the transferred sample components are detected by known means such as optical detection of intercalating dyes, or by detection of radioactivity from hybridized radioactive species, or other known means.

A variety of optical techniques can be used to detect the presence of a target molecule interacting with a probe. For example, if the probe, or probes, are arranged in a linear array within a slot comprising secondary gel, the position and intensity of each signal can be measured by mechanically or optically scanning a single detector along the array of detectable signals. Alternatively, a linear array of detectable signals can be detected by a linear array detector, such as by juxtaposition of the array detector to the array of detectable signals or by optically imaging all or part of the signal array onto the array detector.

When the probes with detectable signals are arranged as a two-dimensional array, a number of detection schemes can be employed. A single detector can be used to measure the signal at each point by mechanical or optical scanning, or by any combination. Alternatively, a linear optical detection array can be used to detect a set of signals by juxtaposition or optical imaging, and multiple sets of such signals can be detected by mechanically or optically scanning the signal array or detector. Alternatively, the two-dimensional array of probes can be optically detected in whole, or in part, by a two-dimensional optical area detector by juxtaposition to, or optical imaging of, the array of optical signals from the immobilized probes.

When the probes are arranged as a three-dimensional array, detection of individual signals be arranged by the above techniques, optionally assisted by first physically taking one or more sub-sections of the array. Alternatively, optical schemes such as confocal microscopic techniques can be employed whereby one or a number of detectable signals are imaged and detected with minimal interference from others, and other signals are subsequently detected after optical adjustment.

The invention further pertains to a diagnostic kit that can be used to detect the presence, or absence, of a target molecule in a biological sample. The diagnostic kit comprises a slotted electrophoresis gel composition comprising a primary gel of matrix material. The primary gel comprises a sample loading zone to facilitate loading of the biological sample and at least one preformed slot-forming an opening for a secondary gel of matrix material. The preformed slot opening refers to a slot that is devoid of primary gel matrix material that formed the primary gel. Optionally, the diagnostic kit provides reagents. These reagents can be specific for particular target molecules. For example, a given reagent can comprise nucleic acid probe specific for a particular bacterial nucleotide sequence which is immobilized to matrix material. This reagent, acting as a secondary gel, can then be prepared, for example, by heating the reagent to form a liquid, and subsequently introduce it into one, or more, preformed slots contained within the primary gel. Multiple reagents can be used to detect the presence, or absence, of multiple target molecules. These multiple reagents once prepared can be introduced into separate preformed slots along the primary gel providing a layered appearance, for example, having the slots aligned perpendicular to the longitudinal axis of the primary gel.

The features and other details of the invention will now be more particularly described and pointed out in the examples. It will be understood that the particular embodiments of the invention are shown by way of illustration and not as limitations of the invention. The principle features of this invention can be employed in various embodiments without departing from the scope of the invention.

### EXAMPLES

### Example 1: Preparation of a Slotted Electrophoresis Gel with Immobilized Oligonucleotides

The casting assembly was prepared by first inserting two spacers (teflon, 0.8 mm in width) between two glass casting plates (10 cm×10 cm), one along one border that is longitudinal to the axis of sample migration and the other along the opposite border. A sample comb (0.8 mm in width) comprising a number of teeth-like projections, in this case ten teeth-like projections, is inserted between the two casting plates along the surface that the sample will be loaded. The sample comb selected will depend upon how many sample are to undergo analysis as well as how sample material is to placed within the sample well. The assembly was secured with tape at each of the four comers. The assembly was placed into a plastic bag and clamped lightly into a Joey™ gel casting apparatus (Owl Scientific, Woburn, Mass.). The apparatus was slightly tilted so that the side containing the sample comb was elevated. A 12% polyacrylamide solution was prepared and polymerization was initiated by adding ammonium persulfate (APS) and N,N,N′,N′-tetra-methylethylene-diamine (TEMED). The polyacrylamide solution was slowly poured into the space created by the casting plates and spacers. The slot-forming comb (0.8 mm in width) was then inserted into the side edge (perpendicular to the sample loading zone) where the slot-forming elements projected across the sample migration pathway. The slot-forming comb had multiple slot-forming elements. The clamps were then securely fastened.

Following polymerization, the assembly was removed from the casting apparatus and plastic bag. The slot-forming comb was removed. The assembly was then placed back into the plastic bag and onto the Joey™ apparatus. A small amount of 12% polyacrylamide solution was poured into the slots to form plugs at the bottom of the slots and were allowed to polymerize.

The immobilized probe layers were then constructed. Polyacrylamide solution containing Acrydite™ (Mosaic Technologies, Boston, Mass.) oligonucleotides was introduced into the slots and allowed to polymerize. The assembly was removed from the apparatus and then from the bag. The slotted electrophoresis gel was then removed from the assembly. The sample comb was removed from the gel. The samples were then loaded and underwent electrophoresis.

### Example 2: Multilayered Slotted Gel for Hybridization Analysis of Multiple Targets Using Gel Immobilized Probes

This example illustrates the use of slotted electrophoresis gels for production of gels containing multiple layers of immobilized hybridization probes, with the layers arranged perpendicular to the direction of sample electrophoresis. Such gels are useful for hybridization analysis of multiple targets in multiple samples.

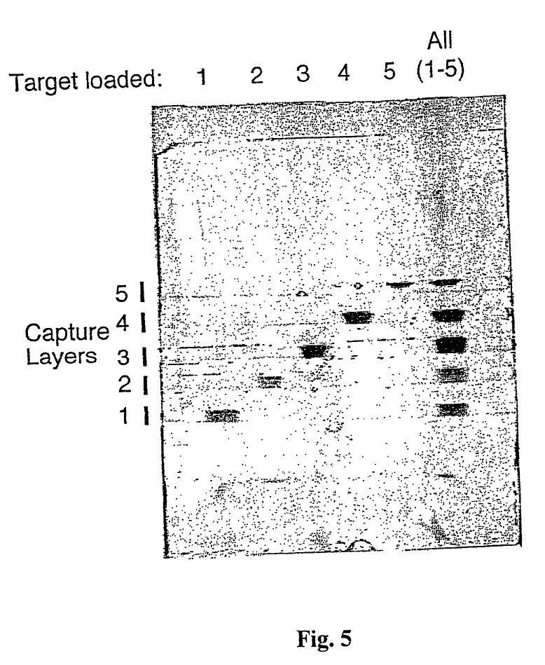

A 10% polyacrylamide gel (containing 1×TBE buffer {89 mM Tris-borate pH 8.3, 2 mM EDTA}) was cast using 10 cm×10 cm front and back glass plates separated by three 0.8 mm (approximately 1-1.5 mm wide) teflon spacers inserted between the plates on the bottom and sides, and five additional slot forming spacers inserted vertically into the gel from above, as illustrated in FIG. 1. The back plate was covered with a sheet of Pagebond film (FMC Bioproducts, Rockland, Me.) to allow the gel to be removed from the glass plates following casting. Following gel polymerization, the slot-forming spacers were removed, and each slot was filled with a 10% acrylamide gel solution containing a different 5′-acrylamide-modified oligonucleotide probe. The 5′-acrylamide modifications were added during automated synthesis using a commercially available acrylamide phosphoramidite (Acrydite™, Mosaic Technologies, Boston, Mass.). The 5′-acrylamide capture probes were added the acrylamide mixtures at a final concentration of 10 μM. After polymerization of the gel sections in the slots, the glass plates were separated, and the gel was removed from the cassette using the Pagebond film support. The gel was trimmed to a size of 10 cm long by 10 cm wide and placed on the bed of a Phast™ horizontal electrophoresis system (Amersham Pharmacia Biotechnology, Piscataway, N.J.). Samples of target oligonucleotides 5′-labeled with fluorescein (targets 1-4, FIG. 5) or CY3 (target 5) were subjected to electrophoresis through the gel at approximately 5V/cm at 25° C. for approximately 1 hour. The target oligonucleotides were designed to be complementary to the capture probes that were immobilized in the layer labeled with the corresponding number. In the right lane, a pool of all five target oligonucleotides was run as a single sample. Approximately 2-5 picomoles of each oligonucleotides target were run in each lane. Following electrophoresis, the gel was scanned with a fluorescence imaging device (Fluorimager, Molecular Dynamics, Sunnyvale, Calif.) using excitation at 485 nm and emmission at 530 nm. (See FIG. 5).

### Example 2. Use of Slotted Electrophoresis Gels for Multiple Sequential Displacement

Six 60-mer polynucleotide sequences were designed and synthesized. Each polynucleotide sequence has a tripartite structure forming a concatemer of three 20 nucleotide sequence motifs. The sequence motifs in the present example are designated D, E, F, cD, cE and cF, wherein “cX” refers to the sequence that is complementary to “X”. Three of the six 60-mers (designated as “Ac” representing 5′-acrylamide modification of the polynucleotide sequence) were synthesized with 5′-acrylamide modifications to allow for immobilization of the polynucleotide sequence within slotted polyacrylamide gels by copolymerization with an acrylamide monomer. The 5′-acrylamide modifications were added during automated synthesis using an acrylamide phosphoramidite (Acrydite™ phosphoramidite, Mosaic Technologies, Boston, Mass.). Immobilizable polynucleotide sequences and complementary displacement (or signal) polynucleotide sequences were hybridized in solution. These displacement polynucleotide sequences when displaced will serve to ultimately displace the signal polynucleotide sequence (E-CY3) which can be detected and is indicative of at least one sample target polynucleotide sequence. The signal polynucleotide sequence was labeled using CY3.

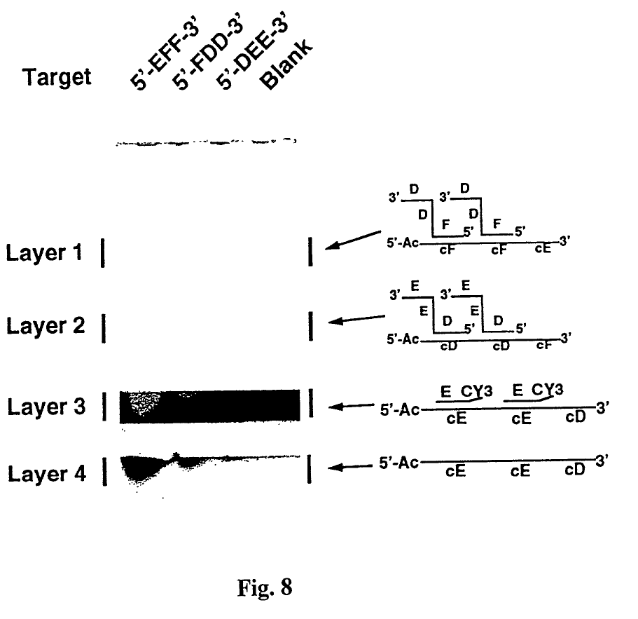

The displacement (or signal) polynucleotide sequences were present in a 4-fold excess in concentration during the hybridization reaction to ensure saturation of all available complementary sites contained within the sequence of the immobilizable polynucleotide sequence. These polynucleotide sequence hybrids were then copolymerized into layers (or slots) in a polyacrylamide slab gel by pouring each copolymer separately into individual slots. The arrangement of the immobilized displacement complexes is shown in FIG. 8.

Hybridization was carried out using 2 μM immobilizable polynucleotide sequence and 8 μM displacement (or signal) polynucleotide sequence in 2×TBE (1×TBE is 89 mM Tris-borate, pH 8.3, 2 mM EDTA). The hybridization reactions were brought to 90° C. and slowly cooled to 40° C., at which temperature the hybridization reactions were performed for an additional 2 hours. Following hybridization, the polynucleotide sequence mixture was mixed 1:1 with 24% acrylamide dissolved in water. Ammonium persulfate and TEMED were added to 0.1% wt/vol and 0.1% vol/vol, respectively. The mixture was poured into horizontal slots (approximately 5 mm wide by 0.8 mm thick) within a precast 12%, 1×TBE polyacrylamide gel for polymerization.

After polymerization of the displacement layers, the gel was subjected to electrophoresis overnight at approximately 2-5 V/cm field gradient in the direction parallel to the long axis of the layers which will serve to remove non-immobilized excess signal and displacement polynucleotide sequences as well as non-immobilized displacement complexes. Following this step, the gel was re-orientated in the apparatus so that samples could be loaded perpendicular to the long axis of the layers and underwent electrophoresis through them sequentially.

The gel contained two probe amplification layers (layer 1 and 2), a probe for generating of a labeled displaced polynucleotide layer (layer 3) and a capture layer to concentrate the labeled displaced polynucleotide into a concentrated band (layer 4). (See FIG. 8). Two pmoles of each of the three different model target polynucleotide sequences, EFF, FDD and DEE, were then loaded into separate lanes of the gel and were subjected to electrophoresis.

Polynucleotide sequence DEE (lane 3) interacted only at the signal conversion layer (layer 3), thereby producing only two displaced signal polynucleotide sequences per target polynucleotide sequence. The FDD polynucleotide sequence (lane 2) was expected to displace two molecules of DEE at the second amplification layer (layer 2), based on affinity preferences, which will ultimately displace four signal polynucleotide sequences per initial target polynucleotide sequence. It is expected that each EFF polynucleotide sequence (lane 1) should displace two molecules from the first layer which will in turn displace four molecules from the second layer and subsequently displace eight molecules from the signal conversion layer. The actual signals obtained at the final layer were quantified using a fluorescence scanner (Molecular Dynamic Fluorimager). The integrated fluorescent signals obtained from the capture layer (layer 4) for the three samples, EFF, FDD and DEE, were 10,400,000, 2,800,000 and 615,000 fluorescence units, respectively. Relative to the DEE signal, the observed signals show a ratio of 16.9(EFF):4.5(FDD): 1(DEE), in qualitative agreement with the predicted trend of 4:2:1, respectively.

### Example 3. Use of Slotted Gel to Perform Two Dimensional Separation with Affinity Capture in the Second Dimension

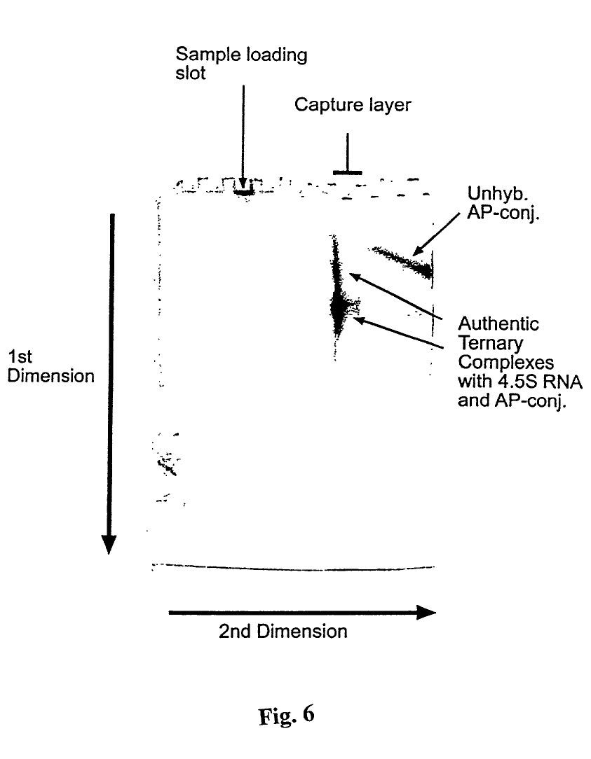

In this example, first dimensional separation (electrophoresis down the gel, in parallel with orientation of slot) takes place without affinity capture. The first dimensional separation of target species occurs based on charge and molecular size. In second dimension, the gel is turned 90° so that the sample undergoes electrophoresis through the which slot contains polyacrylamide with an immobilized hybridization probe. This allows for the identification of “sandwich” ternary complexes that contain both the adaptor and the AP-conjugate which should be efficiently captured by the secondary gel. Unhybridized AP-conjugate will not be captured on the secondary gel layer, and this material appears as a downward sloping line to the right of the capture layer in FIG. 6.

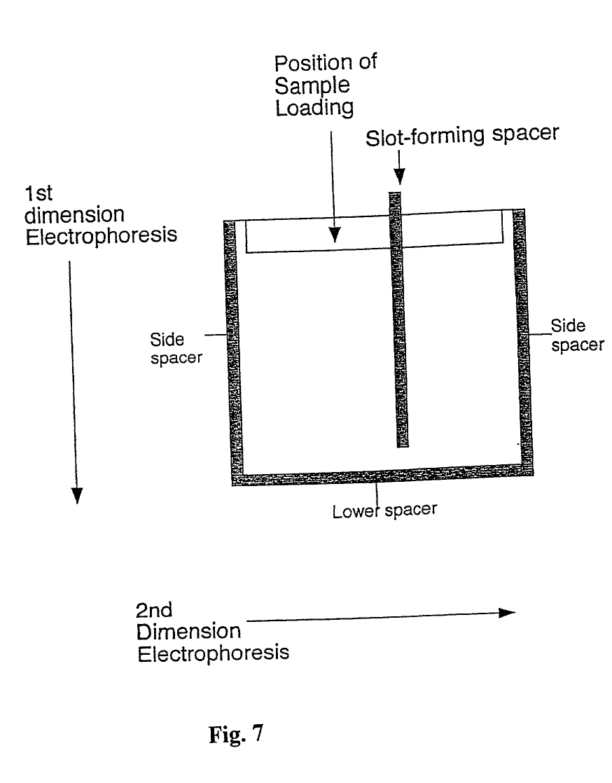

A sandwich probe for detection of E. coli signal recognition particle (SRP) RNA, also known in the literature as 4.5S RNA, used was 12F-APconj: 5′-AP-g gca cac gcg tca tct gcc ttc-3′ [SEQ ID NO 1]; where AP stands for calf intestinal alkaline phosphatase. An adaptor probe complementary to both SRP RNA (on 3′ end of adaptor) and immobilized capture probe (on 5′ end of adaptor) was employed: Adaptor: 5′ -gct gct tcc ttc cgg acc tga-gtg aatac gttcc cgggc ct-3′ [SEQ ID NO. 2]. An immobilized capture probe was immobilized in a slot: 13V: 5′ -acrylamide-ag gcccg ggaac gtatt cac-3′ [SEQ ID NO. 3]. The gel was cast as shown in FIG. 7. The gel (approximately 10 cm×10 cm×0.08 cm, 5% polyacrylamide, 0.5×TBE) was cast with side and lower spacers separating the two glass plates. A fourth spacer was inserted into the middle of the plate sandwich to form the slot. Following polymerization, the central spacer was removed and the slot was filled with acrylamide mixture containing 5% acrylamide, 0.5×TBE, and 5′-acrylamide capture probe (13V) present at 10 μM. After polymerization of the capture layer in the slot, the entire gel was overlaid with additional unmodified 5% polyacrylamide gel and polymerized with a standard multi-well comb inserted to provide sample wells for gel loading.

A single sample was loaded just to the left of the capture slot. The sample was prepared by first hybridizing E. coli total RNA with the Adaptor probe, and then subsequently, hybridizing the 4.5S RNA/Adaptor complex with the 12F AP probe.

Hybridization of E. coli RNA (20 ng) with Adaptor (0.125 μM) in 40 mM Tris-HCl pH 8.3, 0.1 M NaCl was accomplished by incubating the mixture in a thermocycler at 86° C. for 5 minutes, slow cooling the mixture to 65° C. at a rate of 0.5° per minute, then cooling to 20° C. The 12F AP-conjugate probe was added to a final concentration of 70 nM and hybridized to the Adaptor/SRP RNA complex by holding the mixture at 55° C. for 10 minutes, and slow cooling the sample to 35° C. over a period of 1 hour.

The sample was loaded onto the gel and underwent electrophoresis in the first dimension for 30 minutes at 20 V/cm using a gel temperature of 30° C. The gel was turned 90° in the gel apparatus and subjected to electrophoresis in the second dimension for 90 minutes at 20 V/cm at room temperature (approximately 25° C.). Following electrophoresis, the gel was stained for AP activity using a chemifluorescent reagent (Attophos, Lumigen, Boehinger Mannheim, Indianaplois, Ind.), and scanned using a fluorescence imager (Fluorimager, Molecular Dynamics, Sunnyvale, Calif.). (See FIG. 6).

### EQUIVALENTS

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A slotted electrophoresis gel composition comprising a primary gel comprising a first matrix with a sample loading zone and at least one preformed slot comprising an opening for a secondary gel comprising a second matrix, wherein said slot can have any geometric configuration within the primary gel.

2. The electrophoresis gel composition of claim 1, wherein at least one preformed slot is aligned longitudinally to the longitudinal axis of the gel.

3. The electrophoresis gel composition of claim 1, wherein at least one preformed slot is aligned perpendicular to the longitudinal axis of the gel.

4. The electrophoresis gel composition of claim 1, wherein at least one preformed slot is aligned diagonally to the longitudinal axis of the gel.

5. The electrophoresis gel composition of claim 1, wherein said first and second matrices are selected from the group consisting of: polyacrylamide, starch, agarose and combinations thereof.

6. The electrophoresis gel composition of claim 5, wherein said first and second matrices are different.

7. The electrophoresis gel composition of claim 1, wherein at least one preformed slot comprises a secondary gel comprising at least one immobilized molecular probe selected from the group consisting of: nucleic acids, modified nucleic acids, nucleic acid analogs, antibody and antigen binding fragments thereof, protein, polypeptide and peptide.

8. An apparatus for producing a slotted electrophoresis gel composition comprising the following:
(a) at least one slot-forming comb, wherein said slot-forming comb is comprised of at least one slot-forming element;
(b) two plates that serve to constrain the electrophoresis medium when in the liquid phase;
(c) at least one spacer that is positioned between the two plates of (b), and
(d) at least two clamps that secure the two plates of (c) together.

9. A method of detecting the presence, or absence, of one, or more, target molecules in a biological sample using the gel composition of claim 1, comprising the following steps:
(a) immobilizing at least one probe within the secondary gel matrix of at least one slot in the primary gel composition;
(b) introducing the biological sample into the primary gel matrix;
(c) subjecting the electrophoresis gel composition to an electric field such that the target molecule(s) migrates into the secondary gel of at least one slot, wherein the secondary gel contains a probe specific for the target molecule and the target molecule interacts with the immobilized probe, thereby forming an immobilized probe complex, and
(d) detecting the presence of the target molecule/probe complex in the electrophoretic medium,
wherein the detection of at least one target molecule/probe complex is indicative of the presence of at least one target molecule in the biological sample.

10. The method of claim 9, wherein said detection is selected from a group consisting of: radioactivity, luminescence, chemiluminescence, affinity-ligand, enzymatic and fluorescence.

11. The method of claim 9, wherein said target molecule is selected from the group consisting of: proteins, polypeptides, carbohydrates, lipids, deoxyribonucleic acid, ribonucleic acid and combinations thereof.

12. The method of claim 9, wherein said probe is selected from the group consisting of: nucleic acids, modified nucleic acids, nucleic acid analogs, antibody and antigen binding fragments thereof, protein, polypeptide and peptide.

13. A method of detecting the presence, or absence, of one, or more, target molecules in a biological sample using the gel composition of claim 1, comprising the following steps:
(a) forming a secondary gel comprising matrix material different from that comprising the primary gel matrix in at least one slot located in the primary gel composition;
(b) introducing the biological sample into the primary gel matrix;
(c) subjecting the electrophoresis gel composition to an electric field such that the target molecule(s) migrates into the secondary gel of at least one slot, wherein the secondary gel contains a different matrix material from the primary gel composition and the target molecule's migration rate alters, and
(d) detecting the presence of the target molecule in the electrophoretic medium, wherein the detection of at least one target molecule is indicative of the presence of at least one target molecule in the biological sample.

14. The method of claim 13, wherein said detection is selected from a group consisting of: radioactivity, luminescence, chemiluminescence, affinity-ligand, enzymatic and fluorescence.

15. The method of claim 13, wherein said target molecule is selected from the group consisting of: proteins, polypeptides, carbohydrates, lipids, deoxyribonucleic acid, ribonucleic acid and combinations thereof.

16. The method of claim 13, wherein the difference of matrix material between the secondary gel and primary gel is either the composition of matrix material used and/or percentage or matrix material employed.

17. A diagnostic kit for determining the presence, or absence, of a target molecule in a biological sample comprising:
(i) an electrophoresis gel composition comprising a primary gel comprising a first matrix with a sample loading zone and at least one preformed slot opening for a secondary gel, wherein said slot can have any geometric configuration within the primary gel, and
(ii) optionally one, or more, reagent vials comprising one, or more, second matrices comprising reagents specific for the detection of one, or more, target molecules in a biological sample, wherein these reagents are immobilized to a medium.

18. Diagnostic kit reagents comprising matrix material comprising immobilized probes specific for target molecules.

19. The method of claim 9, wherein said target molecule is selected from the group consisting of SEQ ID NO. 1.

20. The method of claim 9, wherein said probe is selected from the group consisting of: SEQ ID NO. 2 and SEQ ID NO. 3.


SEQUENCE LISTING (3 sequences)

SEQ ID NO: 1 - DNA, 22 bp, Artificial Sequence
  alkaline phosphatase conjugated sandwich probe
ggcacacgcgtcatctgccttc

SEQ ID NO: 2 - DNA, 41 bp, Artificial Sequence
  adapter probe
gctgcttccttccggacctgagtgaatacgttcccgggcct

SEQ ID NO: 3 - DNA, 20 bp, Artificial Sequence
  capture probe
aggcccgggaacgtattcac